(19) United States
(12) Patent Application Publication
(10) Pub. No.: US 2007/0112427 A1
(43) Pub. Date: May 17, 2007

(54) Intervertebral Spacer

(75) Inventors: William Christy, Winter Park, FL (US); Mark Goldin, Orlando, FL (US)

Correspondence Address: FROST BROWN TODD, LLC, 2200 PNC CENTER, 201 E. FIFTH STREET, CINCINNATI, OH, 45202, US

(73) Assignee: AOI Medical, Inc., Orlando, FL (US)

(21) Appl. No.: 11/560,001

(22) Filed: Nov. 15, 2006

Related U.S. Application Data
(60) Provisional application No. 60/737,436, filed on Nov. 16, 2005.

(51) Int. Cl. A61F 2/44; A61B 17/70

(52) U.S. Cl. 623/17.11; 606/61; 623/17.16

(57) ABSTRACT

Provided is an intervertebral implant operably configured to maintain limited mobility between adjacent vertebrae. Versions of the intervertebral implant may take the place of an intervertebral disk and may function in a manner substantially similar to an intervertebral disk. In one version, the intervertebral implant includes a spacer having a first surface conducive to bone fusion and a second surface resistant to bone fusion.

### PRIORITY

The application claims priority from the disclosure of U.S. Provisional Patent Application Ser. No. 60/737,436, entitled “Intervertebral Spacer,” filed Nov. 16, 2005, which is herein incorporated by reference in its entirety.

### BACKGROUND

Versions of the present invention relate to retaining spinal mobility following discectomies and other spinal procedures and, more particularly, to retaining spinal mobility following discectomies by providing a replacement disc allowing at least partial spinal movement. A variety of medical devices have been created and used, but no one prior to the inventor(s) has created or used the invention described in the appended claims.

Versions of the present invention relate to retaining spinal mobility following discectomies and other spinal procedures and, more particularly, to retaining spinal mobility following discectomies by providing a replacement disc allowing at least partial spinal movement. A variety of medical devices have been created and used, but no one prior to the inventor(s) has created or used the invention described in the appended claims.

### BRIEF DESCRIPTION OF THE FIGURES

In accordance with versions herein, it is believed the present invention will be better understood from the following description taken in conjunction with the accompanying drawings. The drawings and detailed description that follow are intended to be merely illustrative and are not intended to limit the scope of the invention.

FIG. 1 presents a side view of a spacer positioned between two vertebra having one surface adapted to fuse with a vertebra;

FIG. 2 presents a side view of an alternate version of a spacer positioned between two vertebra having one surface adapted to fuse with a vertebra.

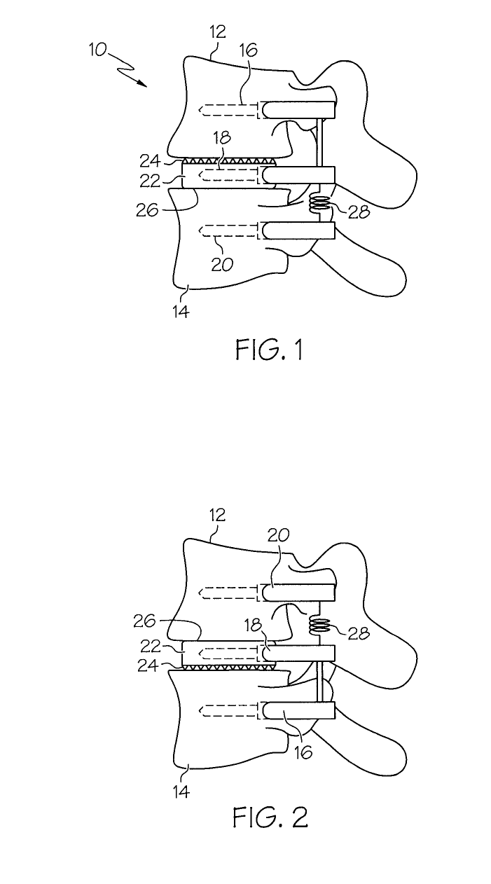

### DETAILED DESCRIPTION OF THE INVENTION

In versions contained herein, the device is an intervertebral implant 10 that is intended to maintain limited mobility between adjacent vertebrae 12, 14 or take the place of a vertebra. In one version, pins 16 and 20 are inserted into the vertebrae of a patient surrounding a disc that is to be removed with, for example, a pedicle screw system having dynamic stabilization. Pin 18 is inserted into the spacer 22. Once inserted, the vertebrae 12, 14 may be held apart while a spacer 22 is placed therebetween. The spacer 22 may be, for example, configured to match the natural shape of the patient's disc.

The spacer 22 includes a first surface 24 adapted to fuse with a first vertebra 12. The first surface 24 may be constructed from any suitable material with which bone may fuse and may be provided with a rough, porous, and/or textured surface to facilitate fusion. The spacer 22 further includes a second surface 26 adapted to lie adjacent to the second vertebra 14. In one version, the second surface 26 is configured such that it is resistant to the fusion of the spacer 22 to the second vertebra 14 where, for example, the second 26 surface may be substantially smooth and/or constructed from a material resistant to bone fusion. The intervertebral implant 10 may be provided with a spring or flexible element 28, an elastic portion, a memory retention portion, a motion dampening portion, and/or a flexible portion operably configured to allow the vertebrae to move relative to one another. The couplings between the pins and/or the spacer may be rigidly fixed, an elastic portion, a spring portion, a memory retention portion, a motion dampening portion, and/or a flexible portion, and combinations thereof.

It will be appreciated that the spacer 22, the first surface 24, and the second surface 26 may be configured from any suitable biomaterial, artificial material, or combinations thereof. It will be appreciated that the first surface and the second surface 26 are disclosed by way of example only, where any arrangement or configuration of surfaces is contemplated. It is further contemplated that the spacer 22 be configured from a cushioning material and/or shock absorbing material to substantially mimic the natural function of a vertebral disc.

With reference to FIG. 2, the surface intended to resist vertebral fusion may be oriented to either the superior surface or the inferior surface of the vertebra, as illustrated. The second surface may be either convex or concave in shape to match and articulate with the mating vertebra.

Providing a first surface and a second surface as disclosed herein may provide the benefits of at least partial spinal motion, or dynamic stabilization, with the benefits of spinal fusion. By fusing the first surface to a vertebra, the spacer may be secured thereto in a permanent fashion. It is further contemplated, however, that the spacer may be retained within the first and second vertebrae with an artificial ligament system.

With the first surface secured, either by fusion to the bone, an artificial ligament system, or the like, the second surface is operably configured to articulate with the movement of the spine. Unlike traditional spinal fusion procedures, where two vertebrae are fused permanently together, versions herein may maintain at least partial mobility between a vertebra and the spacer. This mobility may increase a patient's range of motion and comfort level such that, following such a procedure, the results may feel more natural than traditional spinal fusion.

What is claimed is:

1. A medical apparatus comprising:
(a) a first pin, the first pin being associated with a first vertebra;
(b) a second pin, the second pin being associated with a second vetebra; and
(c) a spacer, the spacer associated with the first pin via a first coupling and with the second pin via a second coupling, wherein the spacer includes a first surface resistant to bone fusion and is configured for retention between the first vertebra and the second vertebra.

2. The medical apparatus of claim 1, wherein the spacer includes a second surface conducive to bone fusion.

3. The medical apparatus of claim 1, wherein the spacer is operably configured to function substantially as a vertebral disc.

4. The medical apparatus of claim 1, wherein at least one of the first coupling and the second coupling includes a flexible element such that the first vertebra and the second vertebra are movable relative to one another.

5. The medical apparatus of claim 1, wherein the first pin and the second pin are inserted into the vertebrae with a pedicle screw system.

6. The medical apparatus of claim 1, wherein the spacer is retained between the first vertebra and the second vertebra with a third pin.

7. The medical apparatus of claim 1, wherein the spacer includes a second surface resistant to bone fusion.

8. The medical apparatus of claim 7, wherein the spacer is retained between the first vertebra and the second vertebra with an artificial ligament.

9. The medical apparatus of claim 1, wherein the spacer comprises a second surface resistant to bone fusion having a configuration selected from the group consisting of convex, concave, including combinations thereof.

10. The medical apparatus of claim 1, wherein the first surface resistant to bone fusion has an orientation selected from the group consisting of facing the superior surface of the first vertebra, facing the inferior surface of the first vertebra, facing the superior surface of the second vertebra, facing the inferior surface of the second vertebra, and combinations thereof.

11. A method for providing spinal mobility follow a discectomy comprising:
providing a medical apparatus comprising;
(a) a first pin, the first pin being associated with a first vertebra;
(b) a second pin, the second pin being associated with a second vetebra; and
(c) a spacer, the spacer associated with the first pin via a first coupling and with the second pin via a second coupling, wherein the spacer includes a first surface resistant to bone fusion and is configured for retention between the first vertebra and the second vertebra;
removing a patient's vertebral disc; and
inserting the medical apparatus.

12. The method of claim 11, wherein the spacer includes a second surface conducive to bone fusion.

13. The method of claim 11, wherein the spacer is operably configured to function substantially as a vertebral disc.

14. The method of claim 11, wherein at least one coupling includes a flexible element such that the first vertebra and the second vertebra are movable relative to one another.

15. The method of claim 11, wherein the first pin and the second pin are inserted into the vertebrae with a pedicle screw system.

16. The method of claim 11, wherein the spacer is retained between the first vertebra and the second vertebra with a third pin.

17. The method of claim 11, wherein the spacer includes a second surface resistant to bone fusion.

18. The method of claim 17, wherein the spacer is retained between the first vertebra and the second vertebra with an artificial ligament.

19. The method of claim 11, wherein the spacer comprises a second surface resistant to bone fusion having a configuration selected from the group consisting of convex, concave, including combinations thereof.

20. The method of claim 11, wherein the first surface resistant to bone fusion has an orientation selected from the group consisting of facing the superior surface of the first vertebra, facing the inferior surface of the first vertebra, facing the superior surface of the second vertebra, facing the inferior surface of the second vertebra, and combinations thereof.
